(19) United States
(12) Patent Application Publication
(10) Pub. No.: US 2013/0005733 A1
(43) Pub. Date: Jan. 3, 2013

(54) COMBINATION ANTI-CANCER THERAPY

(75) Inventors: Sharon Barr, Huntington, NY (US); Elizabeth A. Buck, Huntington, NY (US); David M. Epstein, Huntington, NY (US); Prafulla C. Gokhale, Superior, CO (US); Mark R. Miglarese, Farmingdale, NY (US)

(73) Assignee: OSI Pharmaceuticals, LLC

(21) Appl. No.: 13/583,396

(22) PCT Filed: Mar. 9, 2011
(86) PCT No.: PCT/US11/27673

§ 371 Date: Sep. 7, 2012

Related U.S. Application Data
(60) Provisional application No. 61/311,832, filed on Mar. 9, 2010.

(51) Int. Cl. A61K 31/53; A61P 35/00; A61P 35/04; A61K 31/4985

(52) U.S. Cl. 514/243; 514/249

(57) ABSTRACT

Methods and compositions for treating cancer comprising administering to a patient inhibitors of mT0RC1/C2, IGF-1 R, and IR. In some aspects, a combination of an mT0RC1/C2 inhibitor and an IGF-1 R/IR inhibitor is employed. Other aspects are described herein.

This application claims the benefit of prior U.S. Appl. No. 61/311,832 (filed Mar. 9, 2010), the entire contents of which are incorporated herein by this reference.

### FIELD AND BACKGROUND

The present invention pertains at least in part to anti-cancer treatment, certain chemical compounds and anti-cancer agents, and methods of treating tumors and cancers with the compounds, including with rational combinations thereof.

Dysregulation of signaling pathways mediated by many other kinases is a key factor in the development of human diseases. Aberrant or excessive protein kinase activity or expression has been observed in many disease states including benign and malignant proliferative diseases, disorders such as allergic contact dermatitis, rheumatoid arthritis, osteoarthritis, inflammatory bowel diseases, chronic obstructive pulmonary disorder, psoriasis, multiple sclerosis, asthma, disorders related to diabetic complications, and inflammatory complications of the cardiovascular system such as acute coronary syndrome.

IGF-1R is a transmembrane RTK that binds primarily to IGF-1 but also to IGF-II and insulin with lower affinity. Binding of IGF-1 to its receptor results in receptor oligomerization, activation of tyrosine kinase, intermolecular receptor autophosphorylation and phosphorylation of cellular substrates (major substrates are IRS1 and Shc). The ligand-activated IGF-1R induces mitogenic activity in normal cells and plays an important role in abnormal growth.

IGF-1R performs important roles in cell division, development, and metabolism, and in its activated state, plays a role in oncogenesis and suppression of apoptosis. IGF-1R is known to be overexpressed in a number of cancer cell lines (IGF-1R overexpression is linked to acromegaly and to cancer of the prostate). By contrast, down-regulation of IGF-1R expression has been shown to result in the inhibition of tumorigenesis and an increased apoptosis of tumor cells.

The IGF-1 pathway in human tumor development has an important role: 1) IGF-1R overexpression is frequently found in various tumors (breast, colon, lung, sarcoma) and is often associated with an aggressive phenotype. 2) High circulating IGF1 concentrations are strongly correlated with prostate, lung and breast cancer risk. Furthermore, IGF-1R is required for establishment and maintenance of the transformed phenotype in vitro and in vivo (Baserga R., Exp. Cell. Res., 1999, 253, 1-6). The kinase activity of IGF-1R is essential for the transforming activity of several oncogenes: EGFR, PDGFR, SV40 T antigen, activated Ras, Raf, and v-Src. The expression of IGF-1R in normal fibroblasts induces neoplastic phenotypes, which can then form tumors in vivo. IGF-1R expression plays an important role in anchorage-independent growth. IGF-1R has also been shown to protect cells from chemotherapy-, radiation-, and cytokine-induced apoptosis. Conversely, inhibition of endogenous IGF-1R by dominant negative IGF-1R, triple helix formation or antisense expression vector has been shown to repress transforming activity in vitro and tumor growth in animal models.

Targeting the IGF signaling pathway is a strategy in the development of anti-cancer therapies. The development for use as anti-tumor agents of compounds that directly inhibit the kinase activity of IGF-1R, as well as antibodies that reduce IGF-1R kinase activity by blocking IGF-1R activation or antisense oligonucleotides that block IGF-1R expression, are areas of intense research effort. The highly homologous insulin receptor presents additional complexity in targeting the IGF pathway. IR-A and IGF-1R/IR hybrid activity can have proliferative effects. Ulanet et al., PNAS, 107(24), 10791-10798 (2010). OSI-906 is a small molecule inhibitor of IGF-1R and IR, which is currently in clinical development. US 2006/0235031 describes the preparation and use of OSI-906 (Example 31). See also Mulvihill et al., Future Med. Chem., 1(6), 1153-1171 (2009).

The Tor genes were originally identified in yeast as the targets of the drug rapamycin. The structurally and functionally conserved mammalian counterpart of yeast TOR, mTOR was later discovered. mTOR is a member of the phosphoinositide kinase-related kinase (PIKK) family, but rather than phosphorylating phosphoinositides, phosphorylates proteins on serine or threonine residues. Genetic studies have shown that mTOR is essential for cell growth and development in fruit flies, nematodes and mammals, and the disruption of the genes encoding mTOR results in lethality in all species. Several studies have demonstrated that mTOR has a central role in controlling cell growth, proliferation and metabolism. mTOR regulates a wide range of cellular functions, including translation, transcription, mRNA turnover, protein stability, actin cytoskeletal organization and autophagy. There are two mTOR complexes in mammalian cells. mTOR complex I (mTORC1) is a raptor-mTOR complex, which mainly regulates cell growth in a rapamycin-sensitive manner whereas mTOR complex II (mTORC2) is a rictor-mTOR complex, which regulates cytoskeletal organization in a rapamycin-insensitive manner.

Dysregulation of mTOR pathway is emerging as a common theme in diverse human diseases and as a consequence drugs that target mTOR have therapeutic values. The diseases most clearly associated with deregulation of mTORC1 are tuberous sclerosis complex (TSC) and Lymphangioleiomyomatosis (LAM), both of which are cause by mutations in TSC1 or TSC2 tumor suppressors. Patients with TSC develop benign tumors that when present in brain, however, can cause seizures, mental retardation and death. LAM is a serious lung disease. Inhibition of mTORC1 may help patients with hereditary disorders resulting in overactivation of the mTOR axis, including Peutz-Jeghers cancer-prone syndrome caused by LKB1 mutation and Cowden's disease resulting from loss of PTEN. mTORC1 may also have role in the genesis of sporadic cancers. Inactivation of several tumor suppressors, in particular PTEN, p53, VHL and NF1, has been linked to mTORC1 activation. Rapamycin and its analogues (e.g. CCI-779, RAD001 and AP23573) inhibit TORC1 and have shown moderate anti-cancer activity in phase II clinical trials. However, due to the negative signal from S6K1 to the insulin/PI3K/Akt pathway, it is important to note that inhibitors of mTORC1, like rapalogs, can activate PKB/Akt. If this effect persists with chronic rapamycin treatment it may provide cancer cells with an increased survival signal that may be clinically undesirable. The PI3K/Akt pathway is activated in many cancers. Activated Akt regulates cell survival, cell proliferation and metabolism by phosphorylating proteins such as BAD, FOXO, NF-KB, p21^{Cip1}, p27^{Kip1}, GSK3β and others. Akt might also promote cell growth by phosphorylating TSC2. Akt activation probably promotes cellular transformation and resistance to apoptosis by collectively promoting growth, proliferation and survival, while inhibiting apoptotic pathways. An inhibitor of both mTORC1 and mTORC2 should be beneficial for treatment of tumors with elevated Akt phosphorylation, and should down-regulate cell growth, cell survival and cell proliferation.

Signaling pathways that are upstream and downstream of mTOR are often deregulated in variety of cancers, including breast, lung, kidney, prostate, blood, liver, ovarian, thyroid, GI tract and lymphoma. High levels of dysregulated mTOR (mammalian target of rapamycin) activity are associated with variety of human cancers and several hamartoma syndromes, including tuberous sclerosis complex, the PTEN-related hamartoma syndromes and Peutz-Jeghers syndrome.

Oncogenes including overexpressed or dysregulated receptor tyrosine kinases and constitutively activated mutant receptors activate PI3K-mediated signaling pathways. Additional alterations of the PI3K-mTOR pathway in human cancers include amplification of the p110 catalytic subunit of PI3K, mutation of the p85 PI3K regulatory subunit, loss of PTEN phosphatase function, loss of INPP4B phosphatase function, amplification or mutation of AKT, mutations in TSC1 or TSC2, and overexpression or amplification of eIF4E or S6K1. Mutation or loss of heterozygosity in TSC1 and TSC2 most often give rise to Tuberous Sclerosis (TSC) syndrome. TSC dysregulation is most is frequently associated with hamartomas, although patients with TSC are at risk for malignant renal cancer of clear-cell histology. Although inactivation of TSC might not lead to malignancy per se, deregulation of this pathway seems crucial for angiogenesis in developing malignancies. TSC2 regulates VEGF production through mTOR-dependent and -independent manner.

Rapamycin, a macrolide antifungal antibiotic, is an allosteric inhibitor of the mTORC1 complex both in vitro and in vivo. Everolimus (RAD001, Afinitor) and temsirolimus (CCI-779) are both approved for the treatment of select indications. Temsirolimus (CCI-779) has shown modest anti-tumor activity in Phase II breast, renal carcinoma and mantle cell lymphoma clinical trials. Although rapamycin analogues are in clinical development for cancer as mTOR inhibitors, the clinical outcome with CCI-779 is relatively modest in breast and renal cancer patients. This is probably because rapamycin partially inhibits mTOR function through raptor-mTOR complex (mTORC1). It has been also found that ⅔ of the breast cancer and ½ of renal cancer patients are resistant to rapamycin therapy. Cloughesy et al. demonstrated that half of the GBM patients treated with rapamycin did not respond, and tumor sections from those patients demonstrated hyperactivation of Akt in response to rapamycin, indicating increased mTORC2 complex activity (Cloughesy T F, et al. 2008 Antitumor Activity of Rapamycin in a Phase I Trial for Patients with Recurrent PTEN-Deficient Glioblastoma. PLoS Med 5(1): e8. doi:10.1371/journal.pmed.0050008

With a recent discovery of rictor-mTOR complex (mTORC2) which is involved in phosphorylation of AKT (S473) that is important in regulation of cell survival and modulation of PKCα that plays a major role in regulation of actin cytoskeletal organization in a rapamycin-independent manner, and inhibition of these activities of mTOR is probably important for broader antitumor activity and better efficacy. Therefore, it can be desirable to use an mTOR inhibitor which would inhibit mTORC1 and mTORC2.

It has been demonstrated that certain mTORC1 functions are insensitive to rapapmycin, such as phosphorylation of 4E-BP1 on multiple sites. Furthermore, rapamycin does not acutely inihibit mTORC2 complexes, thus rapamycin provides only partial inhibition of mTOR signaling. Therefore, the use of a direct mTOR kinase inhibitor, which would completely inhibit the function of both mTORC1 and mTORC2, may be required for broader anti-tumor activity and better efficacy.

Efforts are ongoing to identify inhibitors of PI3K, AKT, and mTOR. Courtenay et al., J. Clin. Oncol., 28, 1075-1083 (2010); Workman et al., Cancer Res., 70, 2146-2157 (2010). OSI-027 is a small molecule dual mTORC1/C2 inhibitor, the preparation and use of which was originally described in US 2007/0112005, Example 258. Preferred salts of OSI-027 are described in WO 2009/117482. OSI-027 is presently in clinical development.

Combination therapy is a method that can result in greater efficacy and diminished side effects relative to the use of the therapeutically relevant dose of each agent alone. In some cases, the efficacy of the drug combination is additive (the efficacy of the combination is approximately equal to the sum of the effects of each drug alone), but in other cases the effect is synergistic (the efficacy of the combination is greater than the sum of the effects of each drug given alone).

R. T. Kurmasheva et al. proposed combining inhibitors of mTOR and antibodies that inhibit IGF-1R to treat nonmetastatic rhabdomyosarcoma. ASCO Educ. Book 2008: 460-464. Bertrand et al. report that the effectiveness of an alpha IGF-1R antibody, A12, could be potentiated with small molecule inhibitors of the Ras/Raf/MEK/ERK or PI3K/Akt/mTOR pathways. Leukemia, 20(7):1254-60 (July 2006). X. Wan et al. report that pretreatment of rhabdomyosarcoma cell lines with an anti IGF-1R antibody led to blockade of rapamycin-induced Akt activation. Oncogene, 26, 1932-1940 (2007). Di Cosimo et al. report RAD001 induction of both pAkt and IRS-1 in breast cancer cell lines, and that combinations of RAD001 with IGF-1R tyrosine kinase inhibitors and monoclonal antibodies prevented RAD001-induced pAkt and resulted in superadditive growth inhibition in vitro and in xenografts. J. Clin. Onc., 2007 ASCO Annual Meeting Proceedings Part I. Vol. 25, No. 18S (June 20 Suppl.), 2007:3511.

Rosen et al. reported that AKT inhibition induces a conserved set of RTKs, including HER3, IGF-1R, and IR, in part due to mTORC1 inhibition. Cancer Cell, 19, 58-71 (2011).

ARIAD Pharmaceuticals has announced clinical trials of the mTOR inhibitor deforolimus in combination with MK-0646, an IGF-1R inhibiting antibody in non-small cell lung cancer and other solid tumors. Imclone IMCA12 is believed to be in clinical trials in combination with temsirolimus. Figitumumab, an anti-IGF-1R monoclonal antibody (Pfizer) and the mTOR inhibitor, everolimus (Novartis) have been combined in a Phase I trial, and the best clinical benefit was a partial response. Each of these combination approaches has combined a selective inhibitor of IGF-1R which does not co-target IR, and an allosteric inhibitor of mTORC1 which does not effectively target mTORC2 . WO 2010/120599 refers to treating certain cancers with a combination of an anti-IGF-1R antibody and one of certain specified mTOR inhibitors. WO 2009/126304, at pp. 290-291, states that IGF-1R activation induces PI3K survival pathway and describes a combination of M13-006 and PI-103 in NSCLC and pancreatic cell lines. See also WO 2009/009016; WO 2009/008992; WO 2007/280928.

OSI Pharmaceuticals, Inc., has reported rapamycin sensitizes tumor cells to OSI-906 by promoting enhanced coupling from IGF-1R to PI3K. Buck, et al., International Conference on Molecular Targets and Cancer Therapeutics (Abstract # PR1). Barr et al. presented at the AACR April 2010 meeting the findings that OSI-906 and OSI-027 synergistically inhibited cell proliferation and in some cases synergistically inducedapoptosis. While mTOR is downstream of IGF-1R and IR, we propose that this synergy results from non-overlapping functions of IGF-1R, IR and mTOR. Thus cells that have limited sensitivity to inhibitors of IGF-1R/IR or mTOR as single agents may have greater sensitivity to the combination.

There is a need for improved anti-cancer therapies, molecular targeted therapies and rational combinations, including combinations targeting IR, IGF-1R, and both mTOR complexes, including additive and synergistic combinations.

### SUMMARY

In some aspects, there is provided a method of treating cancer comprising a tumor or tumor metastasis, in a patient comprising administering a therapeutically effective regimen comprising a first active agent that binds to and directly inhibits the catalytic subunits of C1 and C2 mTOR kinases and a second active agent that binds to and directly inhibits the catalytic subunits of IGF-1R and IR, wherein the first and second agents can be administered at the same time or in any sequence.

In some aspects, the present invention concerns a method of treating a cancer comprising a tumor or tumor metastasis mediated at least in part by the PI3K pathway, comprising administering to a patient in need thereof a therapeutically effective regimen comprising one or more active agents that together effectively inhibit IR, IGF-1R, mTOR (both C1 and C2), wherein the mTOR inhibition tends to activate IR and/or IGF-1R signaling or elevate pIGF-1R. In some embodiments, a combination of OSI-906 and OSI-027 is administered.

### DRAWINGS

FIGS. 1A, 1B: Ovarian cell lines exhibit differential sensitivity to OSI-027 and OSI-906. A panel of ovarian cell lines was treated with varying doses of OSI-027 or OSI-906 as single agents, and cell viability was measured at 72 hours after treatment. The effect of each drug on cell proliferation is shown graphically as a fraction of the vehicle-treated control. The two cell lines which express mutant K-Ras are noted, and demonstrate greater sensitivity to IGF-1R/IR inhibition than mTORC1/mTORC2 inhibition. The cell lines which express mutant PI3K or PTEN are noted, and demonstrate greater sensitivity to mTOR inhibition than IGF-1R/IR inhibition.

FIGS. 2A-2D: The combination of OSI-027 and OSI-906 act synergistically to inhibit cell proliferation and induce apoptosis in both KRAS mutant and KRAS wild type cell lines. (2A-2C) The effect of varying doses of OSI-027 alone on cellular proliferation is shown by the dose response curve with closed circles. The dashed line denotes the prediction for the effect of the combination of OSI-027 and OSI-906 if the two drugs were purely additive, as determined by the Bliss algorithm for additivity. The experimental result for the combination of varying doses of OSI-027 and 5 uM OSI-906 is shown by the dose response curve with the open circles. For each of the cell lines, Ovcar 3 (FIG. 2A), MDAH2774 (FIG. 2B) and Ovcar 5 (FIG. 2C), the KRAS mutation status is noted. The combination of OSI-027 and OSI-906 synergistically induces apoptosis in Ovcar 5 cells (FIG. 2D). Apoptosis, as determined by induction of caspase 3/7 activity, was measured 48 hrs after treatment. Apoptosis is expressed as the fold increase in caspase activity relative to DMSO-treated cells. The effect of OSI-027 alone on caspase 3/7 activity is shown by the dose response curve with the closed circles. The mathematical prediction for the combination of OSI-027 and OSI-906, as described above, is shown by the dashed line. The experimental result for the combination of varying doses of OSI-027 and 5 uM OSI-906 on cellular apoptosis is shown by the dose response curve with open circles.

FIGS. 3A-3B: The combination of OSI-027 and OSI-906 provides superior tumor growth inhibition (TGI) in vivo as compared to monotherapy for either single agent. In vivo efficacy study of OSI-027 or OSI-906 as monotherapy and in combination in the H460 lung carcinoma xenografts (FIG. 3A). The effect of various drug treatments on growth of H460 tumors over a 14 day dosing period, dosed once daily (qd) is shown graphically (FIG. 3A). The table (FIG. 3B) summarizes for each treatment: % tumor growth inhibition (% TGI), % regression (% reg.), body weight loss (BWL) as a percent decrease relative to pre-treatment weight, and the number of deaths in a group of 8 animals (Morbidity/Mortality).

FIGS. 4A-4B: Treatment with OSI-027 results in hyperphosphorylation of IR and IGF-1R in both KRASwt and KRAS mutant cell lines. The effect of DMSO, 3 uM OSI-027, 3 uM OSI-906 or the combination of the two agents on phosphorylation of IR (FIG. 4A, Ovcar 3 cells) or IR and IGF-1R (FIG. 4B, MDAH2774 cells) is shown. As previously described, Ovcar 3 cells express wild type K-Ras while MDAH2774 cells express mutant K-Ras. Images of two technical replicates for each receptor, as individual precipitating antibody spots, are shown. Darker spots indicate increased phosphorylated receptor. The pixel density for each sample was calculated relative to DMSO-treated controls and is expressed as a percentage. Black bars indicate the relative pixel density for IR and white bars represent IGF-1R.

FIGS. 5A-5B: Treatment with OSI-027 or erlotinib results in hyperphosphorylation of IGF-1R in both KRASwt and KRAS mutant H460 cell. The effect of DMSO, 3 uM OSI-027 or 3 uM erlotinib on phosphorylation of IGF-1R is shown. Images of the entire spotted antibody array comprising 42 unique receptor tyrosine kinases in duplicate and controls are shown. Positive controls appear as two dark spots at each corner of the array. The spots showing the greatest increase in phosphorylation following treatment with erlotinib or OSI-027 are the pair of technical replicates corresponding to IGF-1R (FIG. 5A). The pixel density for each sample was calculated relative to DMSO-treated controls and is expressed as a percentage. Black bars indicate the relative pixel density for IGF-1R (FIG. 5B).

FIGS. 6A-6B: The combination of OSI-027 and OSI-906 provides greater inhibition of PRAS40 phosphorylation than either single agent. Lysates of cells treated for 2 hours with 300 nM OSI-027, 300 nM OSI-906 or the combination were resolved by SDS PAGE and the effect on phosphorylation of PRAS40, a downstream substrate to Akt, was measured by western blot. Representative images of multiple western blots are shown for phospho-specific PRAS40, total PRAS 40 and B-actin as a loading control (FIG. 6A). The pixel density for phospho-PRAS 40 was quantitated and expressed graphically as a percent of the DMSO-treated control (FIG. 6B).

FIGS. 7A-7B: The combination of OSI-027 and OSI-906 provides greater inhibition of 4E-BP1 phosphorylation than either single agent. Lysates of cells treated for 2 hours with 300 nM OSI-027, 300 nM OSI-906 or the combination were resolved by SDS PAGE and the effect on phosphorylation of 4E-BP1, a downstream effector of the mTOR axis, was measured by western blot. Representative images of multiple western blots are shown for phospho-specific 4E-BP1, total 4E-BP1 and B-actin as a loading control (FIG. 7A). The pixel density for phospho-4E-BP1 was quantitated and expressed graphically as a percent of the DMSO-treated control (FIG. 7B).

FIGS. 8A-8C: The combination of OSI-027 and OSI-906 is synergistic in cell lines derived from a broad array of tumor types. The table summarizes for each cell line: tumor type, KRAS or BRAF mutation status where known (N/D indicates that the status is not known), the EC50 for OSI-027, the maximal growth inhibition of cells cultured in the presence of 10 uM OSI-027 for 72 hours, expressed as a percent of cells treated with DMSO alone, the EC50 for the combination of OSI-027+OSI-906, and the maximal growth inhibition at 72 hours for cells cultured in the presence of 10 uM OSI-027+5 uM OSI-906.

### DETAILED DESCRIPTION

In some aspects, the invention provides use of a therapeutically effective amount of a combination of a first active agent that binds to and directly inhibits the catalytic subunits of C1 and C2 mTOR kinases and a second active agent that binds to and directly inhibits the catalytic subunits of IGF-1R and IR, for the treatment of a comprising a tumor or tumor metastasis in a patient, wherein the first and second agents can be administered at the same time or in any sequence.

In some aspects, the invention provides a method of treating cancer, tumor, or tumor metastasis, in a patient comprising administering a therapeutically effective regimen comprising a first active agent that binds to and directly inhibits the catalytic subunits of C1 and C2 mTOR kinases and a second active agent that binds to and directly inhibits the catalytic subunits of IGF-1R and IR, wherein the first and second agents can be administered at the same time or in any sequence.

According to the invention, the catalytic subunit means the structural peptide unit of the receptor that binds its substrate. Thus, according to the invention, the first and second agents are not monoclonal antibodies. Furthermore, the first agent is not a rapalog or agent that does not inhibit mTORC2.

Without being bound by theory, the active agents cooperate in that the first agent (mTOR) tends to cause pIR and pIGF-1R elevation, whereas the second agent inhibits IR and IGF-1R. Accordingly, partially overlapping involved signaling pathways may be implicated. Thus, in various settings, the combination is synergistic.

In some aspects, cells of the cancer express IGF-1R and insulin receptor (IR). In some aspects, cancer cells of the cancer have activating K-RAS and/or B-RAF gene mutation. For example, K-RAS mutation (e.g., G13D) can lead to increased pIR and pIGF-1R. In some aspects, cancer cells of the cancer have an activating PI3K mutation and/or PTEN loss. In some aspects, the mTOR inhibition by the first active agent tends to or would elevate pIGF-1R and pIR levels in the absence of the second active agent. In some embodiments, the mTOR inhibition is sufficient to avoid elevation of pAkt.

mTOR inhibitors that inhibit mTORC2 in addition to mTORC1 (unlike for example rapamycin, which only inhibits mTORC1), reduce the level of active, phosphorylated AKT in tumor cells, thus reducing tumor cell survival. This is due to the fact that mTORC2 phosphorylates AKT at residue S473, which enhances the activation of AKT by PDK1, thus promoting cell survival. This is advantageous versus mTOR inhibitors that only inhibit mTORC1, such as rapamycin, since in a majority of tumor cells tested, rapamycin is observed to activate AKT, and thus promote tumor cell survival. The latter is thought to have been a significant factor in the disappointing results obtained with rapamycin in the clinic (e.g. see Fan, Q W et al. 2007, Cancer Res. 67(17):7960-7965, attached, at page 7960, second column, lines 17-21). Thus, a combination of an mTOR inhibitor that directly inhibits both mTORC1 and mTORC2, and an IGF-1R kinase inhibitor, may be effective, as each agent should promote inhibition of AKT via complementary mechanisms, and thus tumor cell apoptosis. In addition, both agents should have anti-proliferative effects due to inhibition of protein synthetic pathways, the mTOR inhibitor via both mTORC1 and mTORC2, and the IGF-1R kinase inhibitor via the PI-3 kinase pathway. By contrast, an mTORC1 inhibitor like rapamycin, which activates AKT, could potentially antagonize an IGF-1R kinase inhibitor, which acts at least partially by inhibiting AKT.

Anti-cancer compounds that inhibit mTOR by binding to and directly inhibiting both mTORC1 and mTORC2 kinases have been found to have a number of important advantages over compounds like rapamycin, or its analogues, that only directly inhibit mTORC1. These include (a) superior inhibition of pAkt and concomitant induction of apoptosis in tumor cells, (b) more complete inhibition of phosphorylation of 4E-BP1, which results in greater anti-proliferative effects, (c) inhibition of pAkt (S473) in all tumor cells, thus leading to superior proapoptotic effects (rapamycin inhibits pAkt (S473) in only ˜20% of cancer cell lines), (d) treatment does not increase pAkt (S473) in any cancer cell type tested, and so does not promote tumor cell survival (unlike rapamycin treatment, which increases pAkt (S473) in 65% of cell lines) and (e) anti-proliferative activity in a far broader spectrum of tumor cells. The advantages of an mTOR inhibitor that directly inhibits both mTORC1 and mTORC2 are also discussed in Barr, S. et al. 2009, AACR Annual Meeting, poster #1839 and Yu K. et al. Cancer Res. 69(15): 6232-6240, at p6232 (Introduction) and p6238-9 (Discussion)).

Multiple “mTOR inhibitors that bind to and directly inhibit both mTORC1 and mTORC2 kinases” of varied chemical structure are known to have this functional activity. This indicates that this functionality is not just a particular characteristic of the compounds of Formula I as described in the instant application, and that it is this functionality that is important for performing the methods of the invention, not any particular chemical structure. mTOR inhibitors that bind to and directly inhibit both mTORC1 and mTORC2 kinases” include, for example: (a) PI-103: e.g. Knight, ZA et al. 2006, Cell 125:733-747; (b) NVP-BEZ235: Lui, Q et al. 2009, Drug Discovery Today 6(2): 47-55; (c) XL-765: Lui, Q et al. 2009, Drug Discovery

Today 6(2): 47-55, at page 52; (d) GSK2126458: Lui, Q et al. 2009, Drug Discovery Today 6(2): 47-55; (e) PP242: Lui, Q et al. 2009, Drug Discovery Today 6(2): 47-55, at page 52; (f) KU-0063794: Lui, Q et al. 2009, Drug Discovery Today 6(2): 47-55, at page 52; (g) Wyeth-BMCL-200910096-27: Lui, Q et al. 2009, Drug Discovery Today 6(2): 47-55, at page 53; (h) GDC-0941: Lui, Q et al. 2009, Drug Discovery Today 6(2): 47-55, at page 51; and (i) Formula I OSI compounds, as described in the instant specification, in WO 07/061737. The agents and documents listed above illustrate that a diversity of chemical structures are capable of binding to and directly inhibiting both mTORC1 and mTORC2 kinases, all of which will have the advantages of such a functional activity as described above when used in combination with an IGF-1R kinase inhibitor.

A dual IR/IGF-1R inhibitor and C1/C2 mTOR inhibitor combination is also advantageous over combinations that include other IGF-1R inhibitors that do not also inhibit IR, due to the fact that inhibition of IGF-1R alone, for example with anti-IGF-1R antibodies, has been shown to lead to up-regulation of the IR signal transduction pathway, which can function to promote growth of cancer cells. Buck et al., Mol. Cancer. Ther., 9, 2652-2664 (2010). A dual IR/IGF-1R inhibitor will inhibit activation of the IR signal transduction pathway.

In some aspects, cancer cells of the cancer are sensitive to OSI-906 and insensitive to OSI-027. In some aspects, cancer cells of the cancer are sensitive to OSI-027 and insensitive to OSI-906.

In some aspects, the first and second agents result in synergistic activity.

In some aspects, the method induces apoptosis in cells of the cancer.

In some aspects, the second active agent comprises OSI-906. In some aspects, the first active agent comprises OSI-027.

In some aspects, cancer cells of the cancer include a mesenchymal phenotype.

In some aspects, the method consists of administering OSI-027 and OSI-906. In some aspects, the OSI-027 and the OSI-906 behave synergistically. In some aspects, the OSI-027 and the OSI-906 behave superadditively.

In some aspects, cells of the cancer have dysregulation of the PI3K axis. The skilled artisan understands that dysregulation of a pathway means cellular signaling is inappropriately turned on, often resulting in uncontrolled growth and spread of tumor cells. For a given pathway or axis, one or more of the associated signaling components may be activated. The phosphatidylinositol-3-OH kinase (PI3K) axis, or PI3K/Akt/mTOR axis, is known to the skilled artisan as a signaling cascade that is implicated in various cancers. See, e.g., Bioorg. & Med. Chem., 20, 4308-4312 (2010).

In some aspects, the cancer comprises ovarian cancer, head and neck cancer, breast cancer, colon cancer, pancreatic cancer, small cell lung cancer, non-small cell lung cancer, lymphoma, prostate cancer, renal cell carcinoma, endometrial carcinoma, glioblastoma, Ewing's sarcoma, adrenocortical carcinoma, gastric cancer, multiple myeloma, anaplastic thyroid cancer, or bone metastasis. In some aspects, the cancer comprises ovarian cancer or non-small cell lung cancer.

In some aspects, OSI-906 is administered in an amount of about 0.1 to 20 mg/kg per day on days of administration.

In some aspects, OSI-027 is administered in an amount of about 0.01 to 10 mg/kg per day on days of administration.

In some aspects, the method results in stable disease or tumor regression or partial response for at least about 4, 8, or 16 weeks.

In some aspects, the method further comprises administering at least one additional active anti-cancer agent.

In some aspects, there is provided a kit comprising a container, compositions of OSI-027 and OSI-906, and a package insert comprising instructions for use of the kit to treat cancer comprising a tumor or tumor metastasis.

In some aspects, there is provided a method of treating cancer in a patient having a tumor or tumor metastasis, in which cells thereof have dysregulation of the PI3K pathway, comprising administering a therapeutically effective regimen comprising one or more active agents that together effectively and directly inhibit IR, IGF-1R, and mTOR (both mTORC1 and mTORC2), wherein the mTOR inhibition elevates pIGF-1R and pIR levels.

In some aspects, there is provided use of a therapeutically effective amount of one or more active agents that together effectively and directly inhibit IR, IGF-1R, and mTOR (both mTORC1 and mTORC2), wherein the mTOR inhibition elevates pIGF-1R and pIR levels, to treat a tumor or tumor metastasis in a patient, in which cells thereof have dysregulation of the PI3K axis.

In some aspects, there is provided a pharmaceutical composition comprising a ratio of OSI-906:OSI-027 of about 0.2:1 to about 50:1 of by mass.

In some aspects, the present invention concerns a method of treating a cancer mediated at least in part by the PI3K pathway, comprising administering to a patient in need thereof a therapeutically effective regimen comprising one or more active agents that together effectively inhibit IR, IGF-1R, mTOR (both C1 and C2), wherein the mTOR inhibition tends to activate IR and/or IGF-1R signaling.

In some aspects, the cancer is driven at least in part by both the PI3K and IGF-1 pathways. In some aspects, the mTOR inhibition tends to elevate pIGF-1R in cancer cells of the cancer. In some aspects, the cancer includes cancer cells having activating K-RAS and/or B-RAF gene mutation. In some aspects, cancer cells of the condition include a mesenchymal phenotype.

In some aspects, cancer cells of the condition are relatively insensitive or refractory to one of OSI-027 or OSI-906. In some aspects, cancer cells of the condition are sensitive to OSI-906 relative to OSI-027. In some aspects, cancer cells of the condition are sensitive to OSI-027 relative to OSI-906.

In some aspects, more than one active agent is administered resulting in synergistic activity.

In some aspects, the method induces apoptosis in the cancer cells.

In some aspects, the active agents comprise OSI-906. In some aspects, the active agents comprise OSI-027. In some aspects, the active agents comprise OSI-027 and OSI-906. In some aspects, the OSI-027 and the OSI-906 behave synergistically. In some aspects, the OSI-027 and the OSI-906 behave superadditively.

In some aspects, the OSI-906 and OSI-027 are administered on different days. In some aspects, the OSI-906 is administered in an amount of about 1 to 15 mg/kg per day on days of administration. In some aspects, the OSI-027 is administered in an amount of about 0.2 to 6 mg/kg per day on days of administration.

In some aspects, at least one additional active anti-cancer agent is administered.

### IGF-1R and IR Agents

Inhibition of insulin receptor (IR) and IGF-1R according to the invention can be attained through selective agents or agents that inhibit both kinases. Agents that inhibit IR and/or IGF-1R can be suitable for administration according to the invention. In some embodiments, the active agent is one that holds marketing approval from at least one regulatory authority, having been shown to be safe and effective.

As used herein, the term “IGF-1R kinase inhibitor” refers to any IGF-1R kinase inhibitor known in the art, and includes any chemical entity that, upon administration to a patient, results in inhibition of a biological activity specifically associated with activation of the IGF-1 receptor in the patient, and resulting from the binding to IGF-1R of its natural ligand(s).

Such IGF-1R kinase inhibitors include any agent that can block IGF-1R activation and the downstream biological effects of IGF-1R activation that are relevant to treating cancer in a patient.

Such an inhibitor can act by binding directly to the intracellular catalytic domain of the receptor and inhibiting its kinase activity. Alternatively, such an inhibitor can act by occupying the ligand binding site or a portion thereof of the IGF-1 receptor, thereby making the receptor inaccessible to its natural ligand so that its normal biological activity is prevented or reduced. Alternatively, such an inhibitor can act by modulating the dimerization of IGF-1R polypeptides, or interaction of IGF-1R polypeptide with other proteins, or enhance ubiquitination and endocytotic degradation of IGF-1R. An IGF-1R kinase inhibitor can also act by reducing the amount of IGF-1 available to activate IGF-1R, by for example antagonizing the binding of IGF-1 to its receptor, by reducing the level of IGF-1, or by promoting the association of IGF-1 with proteins other than IGF-1R such as IGF binding proteins (e.g. IGFBP3). IGF-1R kinase inhibitors include but are not limited to low molecular weight inhibitors, antibodies or antibody fragments, antisense constructs, small inhibitory RNAs (i.e. RNA interference by dsRNA; RNAi), and ribozymes. In a preferred embodiment, the IGF-1R kinase inhibitor is a small organic molecule or an antibody that binds specifically to the human IGF-1R.

IGF-1R kinase inhibitors include, for example imidazopyrazine IGF-1R kinase inhibitors, quinazoline IGF-1R kinase inhibitors, pyrido-pyrimidine IGF-1R kinase inhibitors, pyrimido-pyrimidine IGF-1R kinase inhibitors, pyrrolo-pyrimidine IGF-1R kinase inhibitors, pyrazolo-pyrimidine IGF-1R kinase inhibitors, phenylamino-pyrimidine IGF-1R kinase inhibitors, oxindole IGF-1R kinase inhibitors, indolocarbazole IGF-1R kinase inhibitors, phthalazine IGF-1R kinase inhibitors, isoflavone IGF-1R kinase inhibitors, quinalone IGF-1R kinase inhibitors, and tyrphostin IGF-1R kinase inhibitors, and all pharmaceutically acceptable salts and solvates of such IGF-1R kinase inhibitors.

Additional examples of IGF-1R kinase inhibitors include those in WO 05/097800, which describes 6,6-bicyclic ring substituted heterobicyclic protein kinase inhibitors, WO 05/037836, that describes imidazopyrazine IGF-1R kinase inhibitors, WO 03/018021 and WO 03/018022, that describe pyrimidines for treating IGF-1R related disorders, WO 02/102804 and WO 02/102805, that describe cyclolignans and cyclolignans as IGF-1R inhibitors, WO 02/092599, that describes pyrrolopyrimidines for the treatment of a disease which responds to an inhibition of the IGF-1R tyrosine kinase, WO 01/72751, that describes pyrrolopyrimidines as tyrosine kinase inhibitors, and in WO 00/71129, that describes pyrrolotriazine inhibitors of kinases, and in WO 97/28161, that describes pyrrolo [2,3-d]pyrimidines and their use as tyrosine kinase inhibitors, Parrizas, et al., which describes tyrphostins with in vitro and in vivo IGF-1R inhibitory activity (Endocrinology, 138:1427-1433 (1997)), WO 00/35455, that describes heteroaryl-aryl ureas as IGF-1R inhibitors, WO 03/048133, that describes pyrimidine derivatives as modulators of IGF-1R, WO 03/024967, WO 03/035614, WO 03/035615, WO 03/035616, and WO 03/035619, that describe chemical compounds with inhibitory effects towards kinase proteins, WO 03/068265, that describes methods and compositions for treating hyperproliferative conditions, WO 00/17203, that describes pyrrolopyrimidines as protein kinase inhibitors, JP 07/133280, that describes a cephem compound, its production and antimicrobial composition, Albert, A. et al., Journal of the Chemical Society, 11: 1540-1547 (1970), which describes pteridine studies and pteridines unsubstituted in the 4-position, and A. Albert et al., Chem. Biol. Pteridines Proc. Int. Symp., 4th, 4: 1-5 (1969) which describes a synthesis of pteridines (unsubstituted in the 4-position) from pyrazines, via 3-4-dihydropteridines.

In some preferred embodiments, an active agent binds to and directly inhibits the catalytic subunits of IGF-1R and IR.

In the methods of this invention, an IGF-1R kinase inhibitor that inhibits both IGF-1R and IR kinases may be any IGF-1R kinase inhibitor that inhibits both of these receptor-tyrosine kinases, including pharmacologically acceptable salts or polymorphs thereof. In some embodiments, the IGF-1R kinase inhibitor that inhibits both IGF-1R and IR kinases is a small molecule IGF-1R kinase inhibitor. In some embodiments, an IGF-1R kinase inhibitor that inhibits both IGF-1R and IR kinases is a small molecule IGF-1R kinase inhibitor that is ATP-competitive at the kinase catalytic site. In some embodiments, the ratio of the inhibitor's IC50 (as determined using an in vitro biochemical kinase assay, e.g. see Mulvihill, M. J. et al. (2008) Bioorganic & Medicinal Chemistry, Volume 16, Issue 3, 1359-1375) for IGF-1R kinase versus IR kinase (i.e. 1050 IGF-1R:IC50 IR) is within the range 1:10 to 10:1. In other embodiments, the ratio of the inhibitor's IC50 for IGF-1R kinase versus IR kinase are within a range selected from 1:5 to 5:1; 1:3 to 3:1; 1:2 to 1:3; 1:2 to 1:5; or 1:2 to 1:10. In some embodiments, the IGF-1R kinase inhibitor inhibits both IGF-1R and IR kinases, but has little or no significant inhibitory activity against any other kinases in an in vitro biochemical assay.

Examples of kinase inhibitors that inhibit both IGF-1R and IR kinases include have been published in US 2006/0235031. In particular, the compound cis-3-[8-amino-1-(2-phenylquinolin-7-yl)-imidazo[1,5-a]pyrazin-3-yl]-1-methylcyclobutanol, also known as OSI-906, is described therein. As used herein, reference or recitation of OSI-906 includes any salts, solvates, hydrates, and other physical forms, crystalline or amorphous, thereof. OSI-906, which is a selective orally active IGF-1R kinase inhibitor that also has activity against the insulin receptor (IR), is presently in clinical development. Preliminary clinical activity has been reported. OSI-906 can be prepared according to US 2006/0235031, Example 31.

Examples of IGF-1R kinase inhibitors that inhibit both IGF-1R and IR kinases include, but are not limited to: OSI-906, BMS-554417 (Haluska P, et al. Cancer Res 2006;66(1):362-71); BMS 536924 (Huang, F. et al. (2009) Cancer Res. 69(1):161-170)).

Other inhibitors include BMS-754807, BMS-536924, BMS-554417, AG538, A-947864, KW-2450, AXL-1717, XL-228, INSM-18. Other inhibitors include CP-751871, IMC-A12, MK-0646, AMG-479, MEDI-573, BUB-022, rinfabate, rhuMab IGFR, SCH-717454. Other inhibitors include NVP-AEW541, NVP-ADW742. Other inhibitors are cited in US2007/0203143, Hubbard, R. D. et al., Bioorg. Med. Chem. Lett., doi:10.1016/j.bmcl.2009.01.086 (2009). See also US 2010/0048552; WO 2009/158431; WO 2010/0002655; WO 2009/140128; WO 2009/126304; US 2009/0258365; WO 2008/0073687; WO 2009/032668; US 2008/017688; WO 2009/020990; US 2009/0239924; US 2009/0312321; US 2007/0129364; WO 2007/056151; WO 2007/056170; US 2009/0099133; US 2009/0099229; US 2007/0032512; US 2009/0054508; US 2006/0211678; US 2006/0019957; US 2007/0129399; WO 2005/068452.

The skilled artisan will understand how to profile a compound for potential IR and/or IGF-1R activity.

In any of the methods, compositions or kits of the invention described herein, the term “small molecule IGF-1R kinase inhibitor” refers to a low molecular weight (i.e. less than 5000 Daltons; preferably less than 1000, and more preferably between 300 and 700 Daltons) compound that inhibits IGF-1R kinase by binding to the kinase domain of the enzyme.

mTOR Agents

In some preferred embodiments, inhibition of mTOR C1 and C2 can be achieved with any suitable agent that directly inhibits the catalytic activities of both mTORC1 and mTORC2. In some embodiments, the active agent is one that holds marketing approval from at least one regulatory authority, having been shown to be safe and effective.

In some embodiments, an mTORC1 and C2 inhibitor is trans-4-[4-amino-5-(7-methoxy-1H-indol-2-yl)imidazo[5,1-f][1,2,4]triazin-7-yl]cyclohexanecarboxylic acid (also known as OSI-027). Preferred salts of OSI-027, including a tromethamine salt, are described in WO 2009/117482. As used herein, reference or recitation of OSI-027 includes any salts, solvates, hydrates, and other physical forms, crystalline or amorphous, thereof. OSI-027, which is a selective orally active dual inhibitor of the catalytic activities of both mTORC1 and mTORC2, is presently in clinical development. OSI-027 can be prepared according to US 2007/0112005, Example 258.

In some embodiments, an mTORC1 and C2 inhibitor is OXA-01. Inhibition of mTOR by OXA-01 (also known as OSI-950), which has the imidazopyrazine structure shown below, resulted in phosphorylation of pIGF-1R, indicating a rationale for cotargeting mTOR and IGF-1 R with OSI-027 and OSI-906.

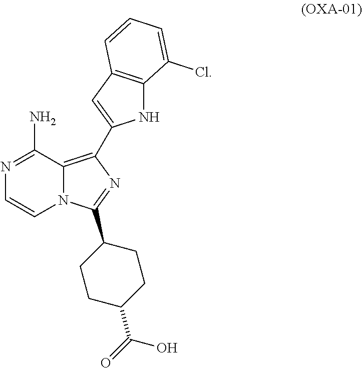 [SMILES: Nc1nccn2c1c(-c1cc3cccc(Cl)c3[nH]1)nc2[C@H]1CC[C@H](C(=O)O)CC1]

In some embodiments, the mTOR inhibitor can be as described in Feldman et al., PLoS Biol., 7(2): e1000038. doi:10.1371/journal.pbio.1000038 (2009), or can be PP-242, PP-30, or derivatives thereof.

Other dual inhibitors include AZD8055, INK-128, Torin-1, and WYE-132. Other agents include GSK-2126458. Other inhibitors are described in: US 2010/0048547; WO2010/006072; US 2009/0312319; US 2010/0015140; US 2007/0254883; US 2007/0149521; Drug Disc. Today Ther. Strateg., 6(2): 47-55 (2009). Other agents can be used rationally as appropriate to supplement the multitarget approach of the present invention. The skilled artisan will understand how to profile a compound for potential mTORC1 and C2 activity.

The present invention further provides any of the methods described herein for treating tumors or tumor metastases, or cancer, in a patient comprising administering to the patient a therapeutically effective amount of an IGF-1R kinase inhibitor that inhibits both IGF-1R and IR kinases, and in addition, simultaneously or sequentially, one or more other cytotoxic, chemotherapeutic or anti-cancer agents, or compounds that enhance the effects of such agents. Such agents may include agents cytotoxic chemotherapeutics, EGFR inhibitors, VEGFR inhibitors, or PDGFR inhibitors that preferably have regulatory or marketing authorization.

### Compositions

In some aspects of the invention, the active agents can be coformulated or separately formulated. For example, OSI-027 and OSI-906 can be coformulated or formulated separately, depending upon the desired dosing approach.

The present invention also provides a pharmaceutical composition comprising an optional pharmaceutically acceptable carrier and/or excipient and, as active ingredient, OSI-906 and/or OSI-027, and optionally one or more other anti-cancer agents. Said pharmaceutical composition can provide synergistic anti-tumor effect.

The above-described pharmaceutical compositions include compositions suitable for oral, rectal, topical, and parenteral (including subcutaneous, intramuscular, and intravenous) administration, although the most suitable route in any given case will depend on the particular host and nature and severity of the conditions for which the active ingredient is being administered. The pharmaceutical compositions may be conveniently presented in unit dosage form and prepared by any of the methods well known in the art of pharmacy.

The active ingredients of the pharmaceutical compositions can be combined in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets, or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the active ingredients of the composition, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

A formulation intended for the oral administration to humans may contain from about 0.5 mg to about 5 g of active agent, compounded with an appropriate and convenient amount of carrier material which may vary from about 5 to about 95 percent of the total composition.

Unit dosage forms will generally contain between from about 1mg to about 2g of the active ingredient, typically 20 mg, 25 mg, 30 mg, 35 mg, 40 mg, 50 mg, 100 mg, 200 mg, 300 mg, 400 mg, 500 mg, 600 mg, 800 mg, or 1000 mg.

In some embodiments, there is provided an oral pharmaceutical composition comprising OSI-906 and OSI-027 in a ratio of about 0.5:1 to about 50:1 of OSI-906:OSI-027 by mass.

In some embodiments, there is provided a kit of parts comprising a container, OSI-027, and OSI-906, and a package insert comprising instructions for use of the kit to treat a tumor or tumor metastasis condition.

Compounds of the invention can be provided for formulation at high purity, for example at least about 90%, 95%, or 98% pure by weight.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared via conventional processing methods. As an example, a cream or ointment is prepared by admixing hydrophilic material and water, together with about 5 wt% to about 10 wt% of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions may also be prepared in powder or liquid concentrate form.

### Patients and Indications

The methods of the present invention can be useful in treating cancer conditions, tumors or tumor metastases, for which inhibition of IR, IGF-1R, mTORC1, and mTORC2 is useful. Such conditions may be mediated or driven at least in part by dysregulation of the IGF and/or PI3K axis, including wherein the mTOR inhibition of the method tends to activate IR and/or IGF-1R signaling.

In some embodiments, the patient to be treated can be insensitive or refractory to treatment with OSI-906 or OSI-027 or other IGF-1R or mTOR inhibitors as a single agents.

In some embodiments, the patient can be a human in need of treatment for cancer, a precancerous condition or lesion, or other forms of abnormal cell growth. The cancer may be, for example: non-small cell lung (NSCL) cancer, breast cancer, colon cancer, pancreatic cancer, lung cancer, bronchioloalveolar cell lung cancer, bone cancer, skin cancer, cancer of the head or neck, cutaneous or intraocular melanoma, uterine cancer, ovarian cancer, rectal cancer, cancer of the anal region, stomach cancer, gastric cancer, uterine cancer, carcinoma of the fallopian tubes, carcinoma of the endometrium, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the ureter, cancer of the kidney, renal cell carcinoma, carcinoma of the renal pelvis, mesothelioma, hepatocellular cancer, biliary cancer, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. The precancerous condition or lesion includes, for example, the group consisting of oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, and precancerous cervical conditions. Preferred embodiments of the cancer/tumor comprise ovarian, prostate, non small cell lung cancer, renal cell carcinoma, endometrial, glioblastoma, lymphoma, or pancreatic cancer.

In some embodiments, the condition comprises ovarian cancer, head and neck squamous cell cancer, breast cancer, colon cancer, pancreatic cancer, small cell lung cancer, non-small cell lung cancer, lymphoma, prostate cancer, renal cell carcinoma, endometrial carcinoma, glioblastoma, Ewing's sarcoma, adrenocortical carcinoma, gastric cancer, multiple myeloma, anaplastic thyroid cancer, or bone metastasis.

In some embodiments, the condition comprises the condition comprises ovarian cancer.

### Methods of Use and Results

It will be appreciated by one of skill in the medical arts that the exact manner of administering treatment according to the invention will be at the discretion of the attending physician. The mode of administration, including dosage, combination with other anti-cancer agents, timing and frequency of administration, and the like, may be affected by the diagnosis of a patient's likely responsiveness, as well as the patient's condition and history. The effectiveness of treatment of any of the methods of treatment described herein can, be determined, for example, by measuring the decrease in size of tumors present in the patients with the neoplastic condition, or by assaying a molecular determinant of the degree of proliferation of the tumor cells.

In some embodiments, OSI-906 and OSI-027 can be co-administered to the patient in the same formulation. In some embodiments, OSI-906 and OSI-027 can be co-administered to the patient in different or separate formulations. In some embodiments, the administration of OSI-906 and OSI-027 to the patient can be simultaneous. In some embodiments, the administration of OSI-906 and OSI-027 to the patient can be sequential.

In conducting the treatment method of the present invention, OSI-906 and OSI-027 can be administered in any effective manner known in the art, such as by oral, topical, intravenous, intra-peritoneal, intramuscular, intra-articular, subcutaneous, intranasal, intra-ocular, vaginal, rectal, or intradermal routes, depending upon the type of cancer being treated, and the medical judgment of the prescribing physician as based, e.g., on the results of published clinical studies.

OSI-906 and OSI-027 can be administered either separately or together by the same or different routes, and in a wide variety of different dosage forms. Both are preferably oral. Both can be administered in single or multiple doses.

In one embodiment, OSI-906 and OSI-027 can be co-administered to the patient by the same route. In another embodiment, OSI-906 and OSI-027 can be co-administered to the patient by different routes.

OSI-906 and OSI-027 can be typically administered to the patient in a dose regimen that provides for the most effective treatment of the cancer (from both efficacy and safety perspectives) for which the patient is being treated, as known in the art, and as disclosed below.

The amount of OSI-906 and OSI-027 administered and the timing of the administration will depend on the type (species, gender, age, weight, etc.) and condition of the patient being treated, the severity of the disease or condition being treated, and on the route of administration. For example, OSI-906 and OSI-027 can each be administered to a patient in doses ranging from about 0.001 to about 100 mg/kg of body weight per day or per week in single or divided doses.

In one embodiment of the above-described methods, OSI-906 and OSI-027 are administered on different days. In another embodiment, neither OSI-906 nor OSI-027 is administered on certain days.

In some embodiments, the treatment method results in stable disease for about 4, 8, 16, 32, or more weeks.

In some embodiments of the above-described methods, the treatments result in tumor size reduction of 5%, 10%, 20%, 30%, 40%, 50%, 60%, 70%, or greater according to RECIST criteria.

### Experimental

Cell lines: Human cancer cell lines were purchased from the American Type Culture

Collection (ATCC). The cell lines Ovcar-3, MDAH2774, Caov3, lgrov-1, and MDA-MB-231 were grown in media as prescribed by the ATCC, containing 10% FCS. HNSCC 1483, HNSCC 1386, HNSCC 1186 were a gift from Memorial Sloan Kettering and were cultured in 1:1 DMEM: Hams F12 with 10% FCS. Ovcar-4, Ovcar-5, and Ovcar-8 were obtained from the NCI and were grown in RPMI with 10% FCS.

Measurement of proliferation: Inhibition of proliferation was measured using the Cell Titer Glo Assay (Promega Corporation, Madison, WI). Cell lines were seeded at a density of 3000 cells per well in a 96-well plate. 24 hours after plating cells were dosed with varying concentrations of drug, either as a single agent or in combination. The signal for Cell Titer Glo was determined 24 hours after dosing and normalized to vehicle-treated controls. Inhibition of proliferation, relative to vehicle-treated controls was expressed as a fraction of 1 and graphed using PRISM® software (Graphpad Software, San Diego, Calif.).

Measurement of apoptosis: Induction of apoptosis as measured by increased Caspase 3/7 activity was determined using the Caspase 3/7 Glo assay (Promega Corporation, Madison, WI). Cell lines were seeded at a density of 3000 cells per well in a 96-well plate. 24 hours after plating cells were dosed with varying concentrations of drug, either as a single agent or in combination. The signal for Caspase 3/7 Glo was determined 24 hours after dosing. The caspase 3/7 activity was normalized to cell number per well, using a parallel plate treated with Cell Titer Glo (Promega Corporation, Madison, WI). Signal for each well was normalized using the following formula: Caspase 3/7 Glo luminescence units/Cell Titer Glo fraction of DMSO control. All graphs were generated using PRISM® software (Graphpad Software, San Diego, Calif.).

Analysis of Synergy: The Bliss additivism model was used to classify the effect of combining OSI-027 and OSI-906 as additive, synergistic or antagonistic. A theoretical curve was calculated for combined inhibition using the equation: E_{bliss}=E_{A}+E_{B}−E_{A}*E_{B}, where E_{A} and E_{B} are the fractional inhibitions obtained by drug A alone and drug B alone at specific concentrations. Here, E_{bliss} is the fractional inhibition that would be expected if the combination of the two drugs was exactly additive. If the experimentally measured fractional inhibition was less than E_{bliss} the combination was said to be synergistic. If the experimentally measured fractional inhibition was greater than E_{bliss} the combination was said to be antagonistic. For dose response curves, the bliss additivity was calculated for varying doses of drug A when combined with a constant dose of drug B. This allowed an assessment as to whether drug B affected the potency of drug A or shifted its intrinsic activity. All plots were generated using Graphpad Prism software.

Measurement of phosphorylated receptor tyrosine kinases: A commercially available membrane-based antibody array (Proteome Profiler Array, R&D Systems, Minneapolis, Minn.) was used to simultaneously co-immunoprecipitate 42 different receptor tyrosine kinases (RTKs) in duplicate and measure specific phospho-epitopes on each. The phospho-epitopes were detected by chemiluminescence, quantitated based on pixel density for each pair of spots and expressed relative to vehicle-treated controls. Pixel density was determined using AlphaEaseFC software (Alpha Innotech, San Leandro, Calif.).

Preparation of Protein Lysates and Western Blotting: Cell extracts were prepared by detergent lysis (50 mM Tris-HCl, pH 8.0, 150 mM NaCl, 1% NP-40, 0.5% sodium deoxycholate, 0.1% SDS, containing protease inhibitor (P8340, Sigma, St. Louis, Mo.) and phosphatase inhibitor (P5726, Sigma, St. Louis, Mo.) cocktails. The soluble protein concentration was determined by micro-BSA assay (Pierce, Rockford, Ill.). Pixel density was determined using AlphaEaseFC software (Alpha Innotech, San Leandro, Calif.).

Protein immunodetection was performed by electrophoretic transfer of SDS-PAGE separated proteins to nitrocellulose, incubation with antibody, and chemiluminescent second step detection (PicoWest; Pierce, Rockford, Ill.). The antibodies included: 4E-BP1, p-4E-BP1(T37/46), PRAS40, and pPRAS40 (T246). Antibodies were obtained from Cell Signaling Technology, Inc. (Danvers, Mass.). For analysis of an agent's effect on the phosphorylation of downstream signaling proteins, cell lines were grown to approximately 80% confluency, at which time the indicated agent was added at the indicated concentration, and cells were incubated at 37° C. for 2 hours. The media was removed, cells were washed two times with PBS, and cells were lysed as previously described.

Evaluation of tumor growth inhibition in vivo: Evaluation of tumor growth inhibition in vivo was conducted at OSI facilities with the approval of the Institutional Animal Care and Use Committee (IACUC) in an American Association for Accreditation of Laboratory Animal Care (AAALAC) accredited vivarium and in accordance with the Institute of Laboratory Animal Research (Guide for the Care and Use of Laboratory Animals, NIH, Bethesda, Md., USA). For this study, female athymic nude nu/nu CD-1 mice (6-8 wks, 20-28 g, Charles River Laboratories, Wilmington, Mass., USA) were allowed to acclimate for a minimum of one week prior to initiation of the study. To evaluate tumor growth inhibition, cells were harvested from cell culture flasks during exponential cell growth, washed twice with sterile PBS, counted and resuspended in PBS to a suitable concentration before subcutaneous implantation on the right flank of nu/nu CD-1 mice. Tumors were established to 200±50 mm³ in size before randomization into treatment groups of 8 mice each.

The TGI study was performed using 20% Trappsol as the vehicle for OSI-027 and 25 mM tartaric acid as the vehicle for OSI-906. Both the drugs were orally administered once-daily for 14 consecutive days. Body weights were determined twice weekly along with tumor volume (V=[length×(width)²]/2) measurements using Vernier calipers. Tumor growth inhibition (TGI) was determined at different time points by the following formula:

$$\% TGI = ( \frac{1 - [ \frac{{T}_{t} / {T}_{0}}{{C}_{t} / {C}_{0}} ]}{1 - [ {C}_{0} / {C}_{t} ]} ) \times 100$$

where Tt=median tumor volume of treated at time t; T0=median tumor volume of treated at time 0; Ct=median tumor volume of control at time t; and C0=median tumor volume of control at time 0. The average TGI over the dosing period was then calculated and reported.

Tumor regressions were determined and calculated as follows using the formula: % Regression=100(W0−Wi)/W0; where W0 is the mean tumor weight for treated group at the initiation of treatment and Wi is the mean tumor weight for that group at time x.

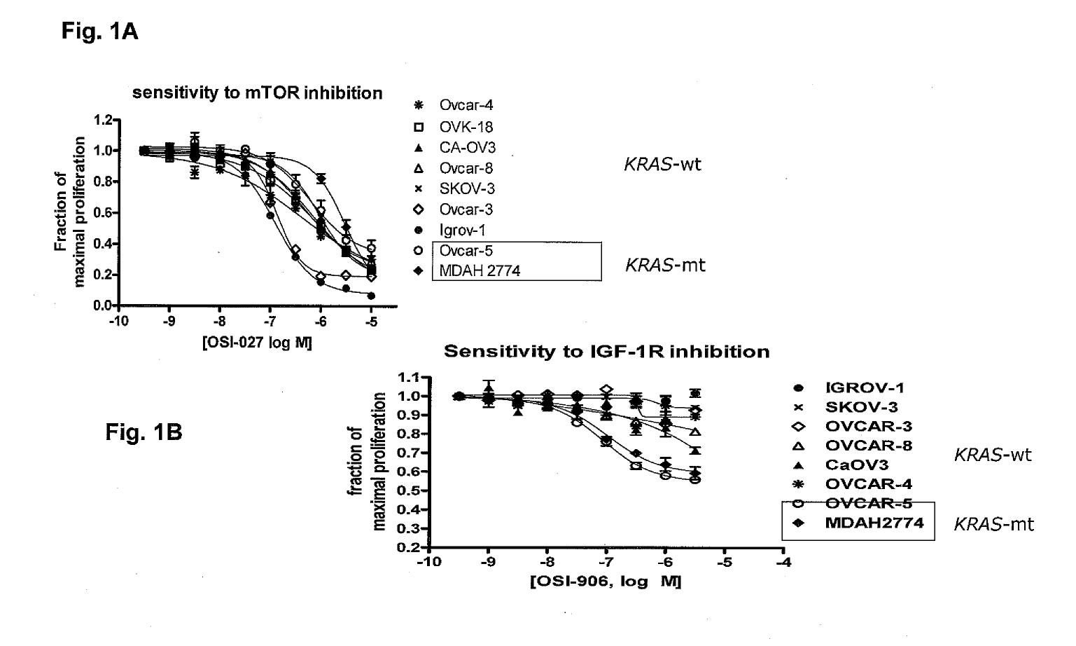

A panel of ovarian cell lines was evaluated for sensitivity to OSI-027, a catalytic site inhibitor of mammalian target of rapamycin (mTOR), or OSI-906, a dual inhibitor of insulin-like growth factor receptor (IGF-1R) and insulin receptor (IR) as single agents. Differential sensitivity in vitro to each of these agents was observed (FIGS. 1A and 1B). Those cell lines which were most sensitive to single agent OSI-027, e.g. Igrov1 and Ovcar3, were among the least sensitive to OSI-906. Conversely, those cell line which were relatively insensitive to OSI-027, e.g. MDAH2774 and Ovcar-5, were the cell lines most sensitive to OSI-906. Notably, those cell lines harboring KRAS mutations, MDAH2774 and Ovcar-5 were sensitive to OSI-906 and not OSI-027. The observation that cell lines exhibited differential sensitivity to OSI-027 and OSI-906 suggested that each of these selective inhibitors may be targeting unique or partially overlapping pathways. We reasoned that combined treatment with OSI-027 and OSI-906 may target the multiple downstream effectors of IGF-1R/IR and mTOR providing synergistic inhibition of proliferation.

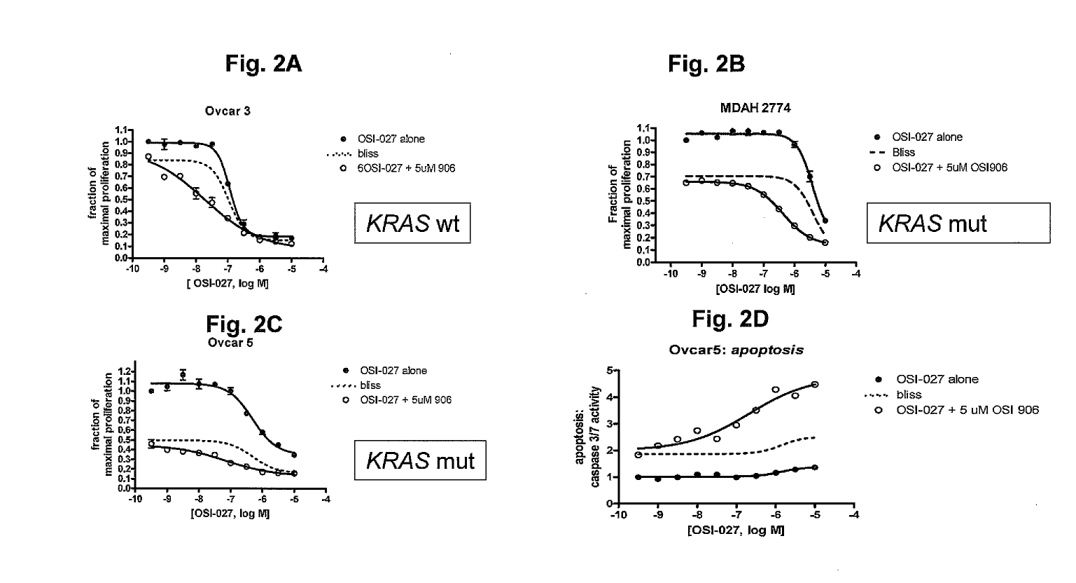

The effect of varying concentrations of OSI-027 on growth inhibition in the presence and absence of OSI-906 is shown in FIGS. 2A-2D. For each cell line, the effect of varying doses of OSI-027 is represented by the closed circles. Synergy was assessed using the bliss additivity model as previously described. The dashed line graphically illustrates the mathematical prediction for the effect of the combination of OSI-027 and OSI-906 if the two drugs were purely additive. The experimental result for the combination of varying doses of OSI-027 and a constant dose of OSI-906 (5 micromolar) is indicated by the open circles. FIGS. 2A-2C demonstrate that the dose-response curve for the combination of OSI-027 and OSI-906 falls significantly below the prediction for additivity, indicating that the combination synergistically inhibits proliferation in both KRASwt and KRAS mutant cell lines. In Ovcar-5 cells, OSI-027 did not significantly induce apoptosis, as measured by induction of caspase 3/7 activity (FIG. 2D, closed circles). The combination of OSI-027 and OSI-906 produced a dose-dependent increase in caspase 3/7 activity (FIG. 2D, open circles) and this induction is significantly greater than the mathematical prediction for additivity (FIG. 2D, dashed line) indicating that the combination of OSI-027 and OSI-906 can act synergistically to induce apoptosis.

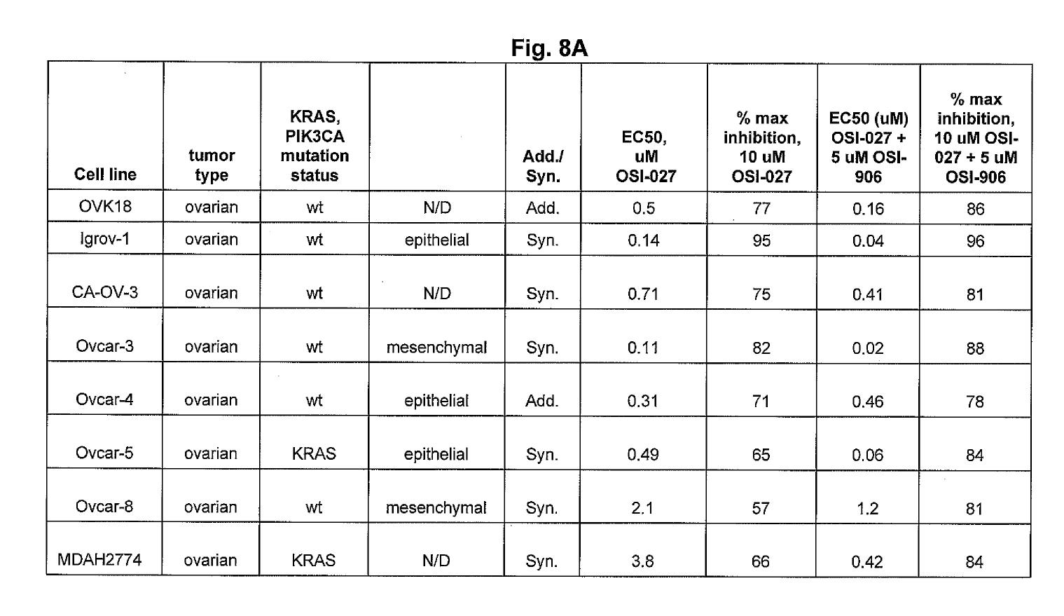

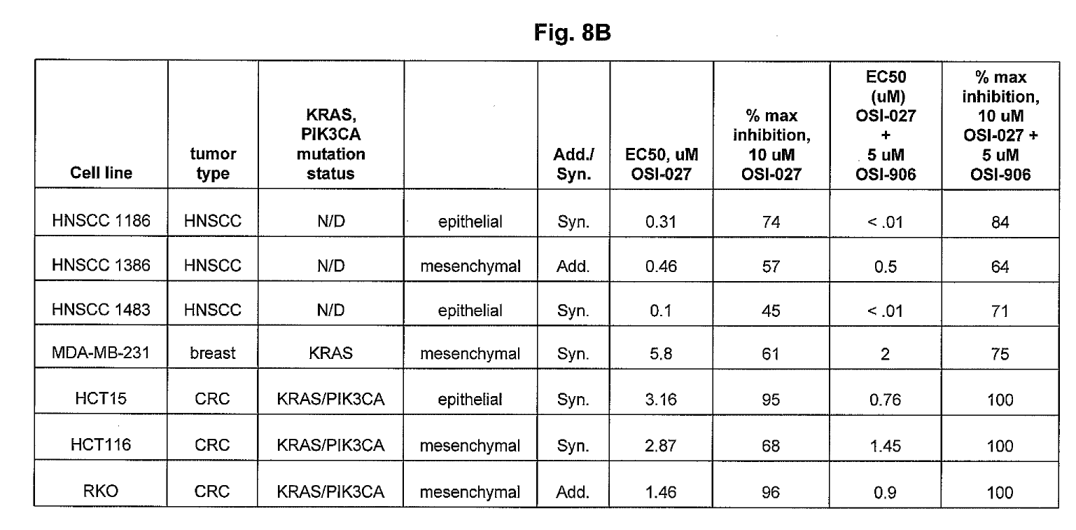

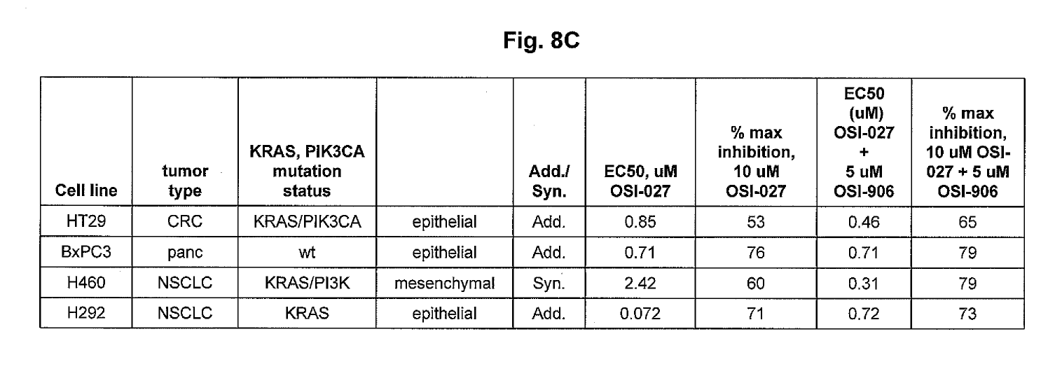

The sensitivities of 15 cell lines derived from ovarian, head and neck squamous cell carcinoma (HNSCC), breast, colorectal (CRC), pancreatic, and non-small cell lung carcinoma (NSCLC) tumors to the combination of OSI-027 and OSI-906 is summarized in FIG. 8. When combined with OSI-906, OSI-027 synergistically inhibits cellular proliferation in the majority of cell lines tested. In every cell line tested, where synergy was observed the combination resulted in a reduced EC50 and frequently improved maximal efficacy. Synergy was assessed using the Bliss additivity model as previously described. In no cell line tested was the combination of OSI-027 and OSI-906 antagonistic.

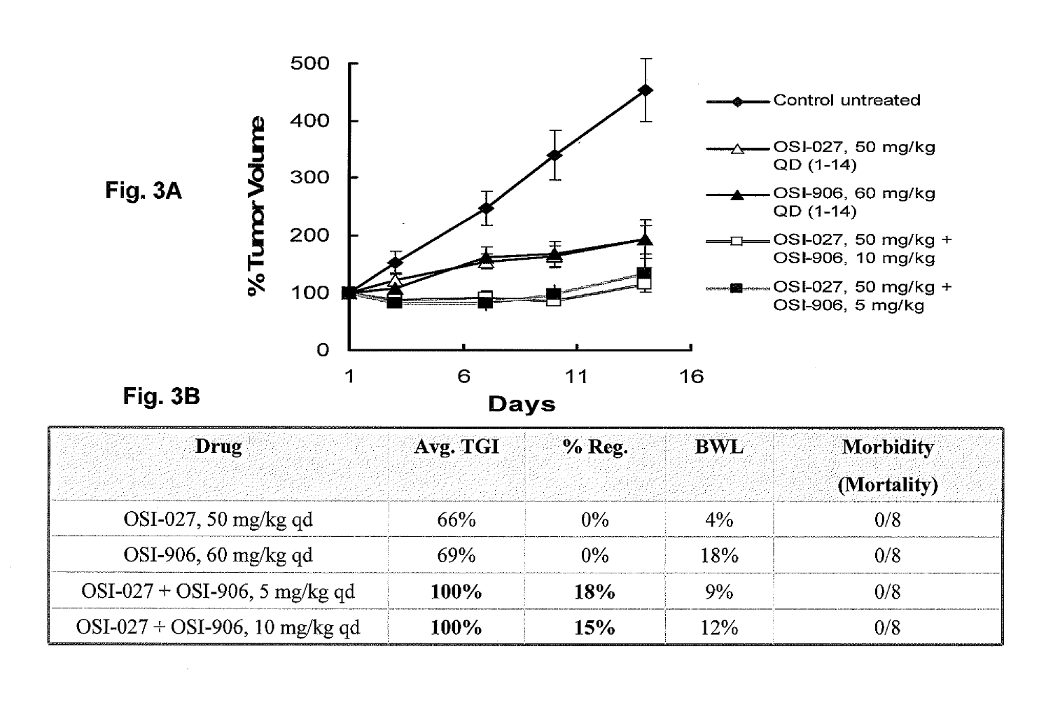

The combination of OSI-027 and OSI-906 was evaluated in vivo in the H460 NSCLC xenograft model. As shown in FIGS. 3A-3B, daily oral administration of OSI-027 at 50 mg/kg for 14 days resulted in modest mean tumor growth inhibition of 66%. OSI-906 as a single agent administered at 60 mg/kg once-daily for 14 days resulted in 69% TGI. In the combination groups, all mice received daily oral administration OSI-027 at 50 mg/kg every day with OSI-906 at 5 mg/kg or 10 mg/kg administered concurrently. Combination of OSI-027 with OSI-906 at 5 mg/kg resulted in significant TGI of 100% (P<0.006 vs. OSI-027 or OSI-906 single agent) with 18% maximum regressions. The combination was well tolerated with an average body weight loss of 9%. Combination of OSI-027 with OSI-906 at 10 mg/kg resulted in similar tumor growth inhibition (100% TGI, P<0.001 vs. OSI-027 or OSI-906 single agent) and regression (15%). This combination was tolerated with an average 12% body weight loss. These data indicate that the combination of OSI-027 with a low dose of OSI-906 provides superior tumor growth inhibition and tumor regression relative to either single agent at the maximal tolerated dose.

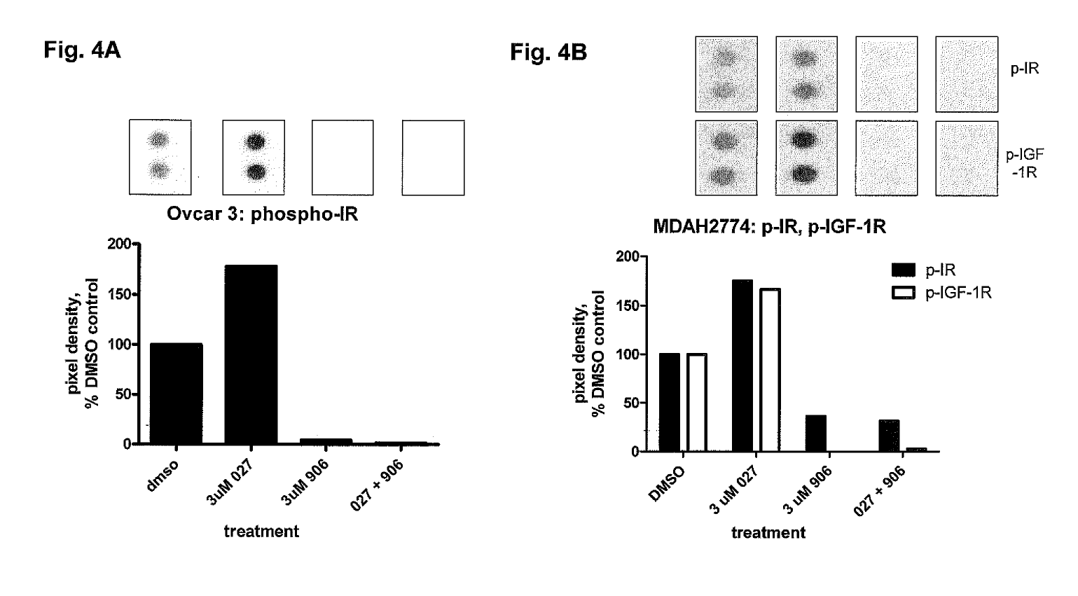

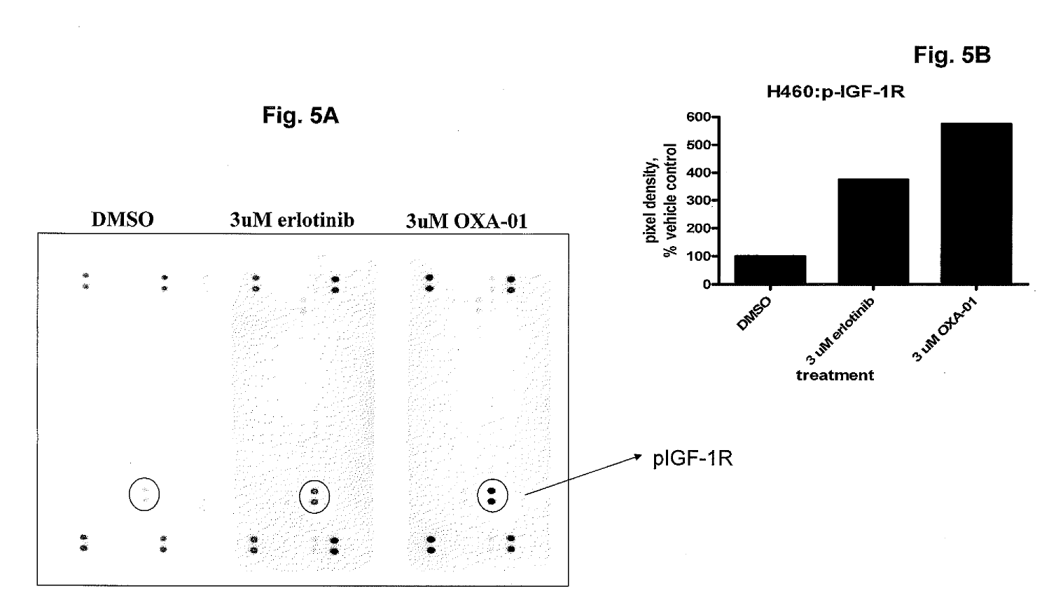

Evaluation of the activation state of a panel of receptor tyrosine kinases indicates a mechanism by which a catalytic site inhibitor of mTOR, such as OSI-027 or OXA-01, sensitizes cells to the effects of an IGF-1R or IR inhibitor such as OSI-906. As shown in FIG. 4A, immunoprecipitation of IR followed by detection with a pan-anti-phosphotyrosine antibody indicates that in Ovcar-3 ovarian carcinoma cells treated with OSI-027, IR is hyperphosphorylated. This elevated phosphorylation state is an indicator of increased activity for both IGF-1R and IR (Lopaczynski et al., 2000; Baserga et al., 1999). Images of the immunoprecipitation reactions, run as panel of 42 different phosphorylated receptor tyrosine kinases on a spotted antibody array, each as a pair of technical replicates, are shown. Pixel density was calculated and is shown graphically in the bar graph below as a percentage of the vehicle-treated control. OSI-906, a selective inhibitor of IR and IGF-1R fully attenuates phosphor-IR, below the basal level detected in the DMSO-treated sample. The combination of OSI-027 and OSI-906 also results in complete reduction of p-IR. Similarly, in MDAH2774 ovarian carcinoma cells (FIG. 4B) the phosphorylation of both IGF-1R and IR are increased following 2 hour treatment with OSI-027, and this is reduced by treatment with OSI-906 or the combination of OSI-027 and OSI-906. Synergistic inhibition of proliferation has been observed in both Ovcar3 and MDAH2774 cells. This effect upon IR and IGF-1R phosphorylation is not unique to OSI-027 but is also observed in H460 lung carcinoma cells treated with OXA-01 (FIGS. 5A, 5B). Erlotinib, a selective EGFR inhibitor, has previously been shown to elevate phosphor-IGF-1R via a compensatory signaling mechanism (Buck et al., 2008) and here we demonstrate that OXA-01 treatment increases p-IGF-1R to a greater degree than erlotinib in this cellular model.

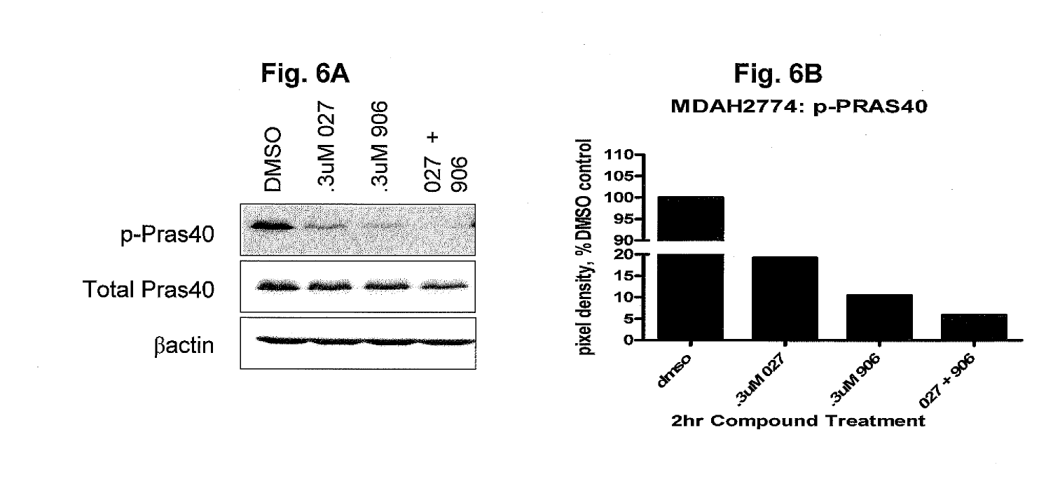

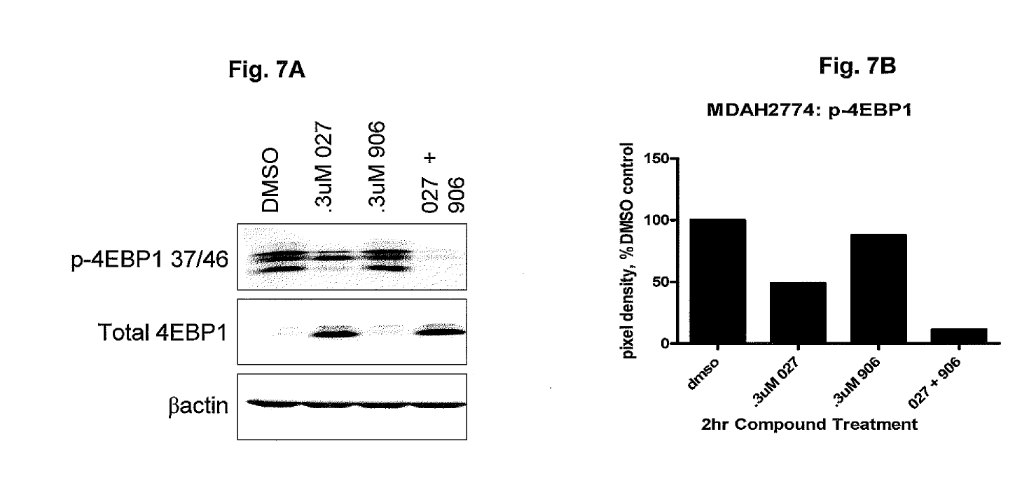

We hypothesized that the combination of OSI-027 and OSI-906 would provide better inhibition of downstream effectors in the Akt signaling axis. In MDAH2774, an ovarian cancer cell line relatively insensitive to OSI-027, treatment with 300 nM OSI-027 results in partial inhibition of phosphor-PRAS40, a direct substrate of Akt and a robust measure of Akt activation (FIG. 6A). OSI-906 inhibits phosphor-PRAS40 to a greater degree and the combination of OSI-027 and OSI-906 provides greater inhibition than either single agent. Band density was calculated and is shown as a bar graph (FIG. 6B). 4E-BP1 is a downstream effector of mTOR signaling and a key regulator of CAP-dependent protein translation. Phosphorylation of 4E-BP1 is reduced by OSI-027 treatment (FIGS. 7A-7B). No significant decrease is observed with OSI-906 treatment, however the combination of OSI-027 and OSI-906 results in superior inhibition of p4E-BP1 as compared to either single agent.

The combination of an mTOR inhibitor such as OSI-027 and inhibitor of IGF-1R or IR such as OSI-906 can act synergistically to inhibit proliferation in vitro in cell lines derived from multiple tumor types. These agents can act synergistically to induce tumor cell apoptosis, and that the combination is superior to monotherapy for either agent even at a significantly reduced dose. Treatment with OSI-027 or OXA-01, both catalytic site inhibitors of mTOR, as well as erlotinib, a selective EGFR inhibitor, can result in increased activation of the IGF-1 receptor or insulin receptor and when this occurs then synergistic inhibition of proliferation is also observed. These data support the concept that treatment with an agent which increases phosphorylation of IGF-1R or IR sensitizes cells to the effects of OSI-906. Consistent with this, we have provided evidence that the combination of OSI-027 and OSI-906 results in greater inhibition of downstream effectors of the Akt/mTOR signaling axis than either single agent, consistent with the synergistic inhibition of proliferation observed.

References: Lopaczynski, W. et al. (2000) Biochem. Biophys. Res. Commun., 279, 955-960; Baserga, R. et al. (1999) Exp. Cell Res. 253, 1-6; Buck et al. (2008) Cancer Res. 68, 8322-32.

### DEFINITIONS

The language and terms herein are to be given their broadest meaning accepted by the skilled artisan, unless otherwise specified.

The term “cancer” in an animal, including human, refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may circulate in the blood stream as independent cells, such as leukemic cells.

“Cell growth”, as used herein, for example in the context of “tumor cell growth”, unless otherwise indicated, is used as commonly used in oncology, where the term is principally associated with growth in cell numbers, which occurs by means of cell reproduction (i.e. proliferation) when the rate of the latter is greater than the rate of cell death (e.g. by apoptosis or necrosis), to produce an increase in the size of a population of cells, although a small component of that growth may in certain circumstances be due also to an increase in cell size or cytoplasmic volume of individual cells. An agent that inhibits cell growth can thus do so by either inhibiting proliferation or stimulating cell death, or both, such that the equilibrium between these two opposing processes is altered.

“Tumor growth” or “tumor metastases growth”, as used herein, unless otherwise indicated, is used as commonly used in oncology, where the term is principally associated with an increased mass or volume of the tumor or tumor metastases, primarily as a result of tumor cell growth.

“Abnormal cell growth”, as used herein, unless otherwise indicated, refers to cell growth that is independent of normal regulatory mechanisms (e.g., loss of contact inhibition).

This includes the abnormal growth of: (1) tumor cells (tumors) that proliferate by expressing a mutated tyrosine kinase or over-expression of a receptor tyrosine kinase; (2) benign and malignant cells of other proliferative diseases in which aberrant tyrosine kinase activation occurs; (4) any tumors that proliferate by receptor tyrosine kinases; (5) any tumors that proliferate by aberrant serine/threonine kinase activation; and (6) benign and malignant cells of other proliferative diseases in which aberrant serine/threonine kinase activation occurs.

The term “treating” as used herein, unless otherwise indicated, means reversing, alleviating, inhibiting the progress of, or preventing, either partially or completely, the growth of tumors, tumor metastases, or other cancer-causing or neoplastic cells in a patient. The term “treatment” as used herein, unless otherwise indicated, refers to the act of treating.

The phrase “a method of treating” or its equivalent, when applied to, for example, cancer refers to a procedure or course of action that is designed to reduce or eliminate the number of cancer cells in an animal, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an animal, is nevertheless deemed an overall beneficial course of action.

As used herein, “agent” or “biologically active agent” refers to a biological, pharmaceutical, or chemical compound or other moiety. Non-limiting examples include simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize that there is no limit as to the structural nature of the agents of the present invention.

The term “agonist” as used herein refers to a compound having the ability to initiate or enhance a biological function of a target protein, whether by inhibiting the activity or expression of the target protein. Accordingly, the term “agonist” is defined in the context of the biological role of the target polypeptide. While preferred agonists herein specifically interact with (e.g. bind to) the target, compounds that initiate or enhance a biological activity of the target polypeptide by interacting with other members of the signal transduction pathway of which the target polypeptide is a member are also specifically included within this definition.

The terms “antagonist” and “inhibitor” are used interchangeably, and they refer to a compound having the ability to inhibit a biological function of a target protein, whether by inhibiting the activity or expression of the target protein. Accordingly, the terms “antagonist” and “inhibitors” are defined in the context of the biological role of the target protein. While preferred antagonists herein specifically interact with (e.g. bind to) the target, compounds that inhibit a biological activity of the target protein by interacting with other members of the signal transduction pathway of which the target protein is a member are also specifically included within this definition. A preferred biological activity inhibited by an antagonist is associated with the development, growth, or spread of a tumor, or an undesired immune response as manifested in autoimmune disease.

An “anti-cancer agent”, “anti-tumor agent”, or “chemotherapeutic agent” refers to any agent useful in the treatment of a neoplastic condition. One class of anti-cancer agents comprises chemotherapeutic agents. “Chemotherapy” means the administration of one or more chemotherapeutic drugs and/or other agents to a cancer patient by various methods, including intravenous, oral, intramuscular, intraperitoneal, intravesical, subcutaneous, transdermal, buccal, or inhalation or in the form of a suppository.

The term “effective amount” or “therapeutically effective amount” refers to that amount of a compound described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g. reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

The term “selective inhibition” or “selectively inhibit” as applied to a biologically active agent refers to the agent's ability to selectively reduce the target signaling activity as compared to off-target signaling activity, via direct or interact interaction with the target.

For purposes of the present invention, “co-administration of and “co-administering” an refer to any administration of the two active agents, either separately or together, where the two active agents are administered as part of an appropriate dose regimen designed to obtain the benefit of the combination therapy. Thus, the two active agents can be administered either as part of the same pharmaceutical composition or in separate pharmaceutical compositions. The OSI-906 can be administered prior to, at the same time as, or subsequent to administration of the OSI-027, or in some combination thereof.

The term “method for manufacturing a medicament” or “use of for manufacturing a medicament” relates to the manufacturing of a medicament for use in the indication as specified herein, and in particular for use in tumors, tumor metastases, or cancer in general. The term relates to the so-called “Swiss-type” claim format in the indication specified.

In the context of this invention, the sensitivity of tumor cell growth to the IGF-1R kinase inhibitor OSI-906 is defined as high (“sensitive”) if the tumor cell is inhibited with an EC₅₀ (half-maximal effective concentration) of less than 1 μM, and low (i.e. resistant) if the tumor cell is inhibited with an EC₅₀ of greater than 10 μM. Sensitivities between these values are considered intermediate. With other IGF-1R kinase inhibitors that inhibits both IGF-1R and IR kinases, particularly compounds of Formula I as described herein, a qualitatively similar result is expected since they inhibit tumor cell growth by inhibiting the same signal transduction pathway, although quantitatively the EC₅₀ values may differ depending on the relative cellular potency of the other inhibitor versus OSI-906. Thus, for example, the sensitivity of tumor cell growth to a more potent IGF-1R kinase inhibitor than OSI-906 would be defined as high when the tumor cell is inhibited with an EC₅₀ that is correspondingly lower. In tumor xenograft studies, using tumor cells of a variety of tumor cell types that all have high sensitivity to OSI-906 in culture in vitro, the tumors are consistently inhibited in vivo with a high percentage tumor growth inhibition (TGI) (See Experimental section herein). In contrast, in similar studies, using tumor cells that have low sensitivity to OSI-906 in culture in vitro, the tumors are inhibited in vivo with only a low percentage tumor growth inhibition (TGI). These data indicate that sensitivity to IGF-1R kinase inhibitors such as OSI-906 in tumor cell culture is predictive of tumor sensitivity in vivo.

In the context of this invention, the sensitivity of tumor cell growth to the OSI-027 is defined as high (“sensitive”) if the tumor cell is inhibited with an EC₅₀ (half-maximal effective concentration) of less than 1 μM, and low (i.e. resistant) if the tumor cell is inhibited with an EC₅₀ of greater than 10 μM. Sensitivities between these values are considered intermediate.

The term EC₅₀ (half maximal effective concentration) refers to the concentration of compound which induces a response halfway between the baseline and maximum for the specified exposure time, and is used as a measure of the compound's potency.

1-24. (canceled)

25. A method of treating a cancer tumor or tumor metastasis in a patient comprising administering together or sequentially a therapeutically effective amount of a first active agent that binds to and directly inhibits the catalytic subunits of C1 and C2 mTOR kinases and a second active agent that binds to and directly inhibits the catalytic subunits of IGF-1R and IR.

26. The method of claim 25, wherein cells of the cancer express IGF-1R and insulin receptor (IR).

27. The method of claims 26, wherein cancer cells of the cancer have at least one of activating K-RAS or B-RAF gene mutation.

28. The method of claim 26, wherein cancer cells of the cancer have at least one of an activating PI3K mutation or PTEN loss.

29. The method of claim 26, wherein cancer cells of the cancer are sensitive to OSI-906 and insensitive to OSI-027.

30. The method of claim 26, wherein cancer cells of the cancer are sensitive to OSI-027 and insensitive to OSI-906.

31. The method of claim 25, wherein the first and second agents behave synergistically.

32. The method of claim 25, which induces apoptosis in cells of the cancer.

33. The method of claim 25, wherein the second active agent comprises OSI-906.

34. The method of claim 25, wherein the first active agent comprises OSI-027.

35. The method of claim 25, consisting of administering OSI-027 and OSI-906.

36. The method of claim 35, wherein the OSI-027 and the OSI-906 behave synergistically.

37. The method of claim 35, wherein the 051-027 and the OSI-906 behave superadditively.

38. The method of claim 25, wherein the cancer comprises ovarian cancer, head and neck cancer, breast cancer, colon cancer, pancreatic cancer, small cell lung cancer, non-small cell lung cancer, lymphoma, prostate cancer, renal cell carcinoma, endometrial carcinoma, glioblastoma, Ewing's sarcoma, adrenocortical carcinoma, gastric cancer, multiple myeloma, anaplastic thyroid cancer, or bone metastasis.

39. The method of claim 25, wherein the cancer comprises ovarian cancer or non-small cell lung cancer.

40. The method of claim 35, wherein OSI-906 is administered as the second agent in an amount of about 1 to 15 mg/kg per day on days of administration.

41. The method of claim 35, wherein OSI-027 is administered as the first agent in an amount of about 0.2 to 6 mg/kg per day on days of administration.

42. The method of claim 25, which results in stable disease or tumor regression for at least about eight weeks.
